(19) United States
(12) Patent Application Publication
(10) Pub. No.: US 2003/0031648 A1
(43) Pub. Date: Feb. 13, 2003

(54) Vectors for expressing heterologous peptides at the amino-terminus of potyvirus coat protein, methods for use thereof, plants infected with same and methods of vaccination using same

(75) Inventors: Tzahi Arazi, Kiryat-Ono (IL); Yoel Moshe Shiboleth, D.N. Hefer (IL); Amit Gal-On, Ramat Hasharon (IL)

Correspondence Address: DR. MARK FRIEDMAN LTD., c/o Bill Polkinghorn, Discovery Dispatch, 9003 Florin Way, Upper Marlboro, MD, 20772, US

(73) Assignee: VIROGENE LTD.

(21) Appl. No.: 09/963,761

(22) Filed: Sep. 27, 2001

Related U.S. Application Data
(60) Provisional application No. 60/253,136, filed on Nov. 28, 2000.

(51) Int. Cl. A61K 48/00; C12N 7/00; A01H 5/00; C12N 15/86

(52) U.S. Cl. 424/93.2; 800/290; 435/235.1; 435/320.1

(57) ABSTRACT

A recombinant vector for expressing a heterologous peptide at the amino-terminus of a potyvirus coat protein. The vector includes sufficient potyvirus nucleic acid sequence to permit viral replication and spread within a plant infected by the vector. The vector further includes a heterologous nucleic acid sequence inserted at the amino-terminus of the potyvirus coat protein. Further disclosed are methods for transiently expressing the heterologous protein in a plant using the vector and plants transiently expressing the vector. Additionally, methods for vaccination which employ the vector to express an antigenic determinant in a plant are disclosed.

This application claims priority from U.S. provisional patent application 60/253,136 filed on Nov. 28, 2000.

### FIELD AND BACKGROUND OF THE INVENTION

The present invention relates to vectors for expressing heterologous Peptides at the amino-terminus of Potyvirus Coat Protein, methods for use thereof, plants infected with same and methods of vaccination using same. More particularly, the present invention relates to a Zucchini Yellow Mosaic Potyvirus (ZYMV) vector capable of expressing at least a portion of a heterologous peptide on the surface of virions so that isolated virions or a portion of a plant, for example a cucurbit fruit, infected therewith may be used as a source of material for vaccination, pharmaceutical or diagnostic applications.

Zucchini yellow mosaic virus is a member of the potyviridae family (Shukla et al. (1989) Adv. Virus Res. 36:273-314.). Potyviridae is the largest group of plant-infecting viruses and its members infect most commercial or cultivated crops.

Worldwide, ZYMV is one of the most devastating diseases of cucurbit species (e.g., squash, melon, watermelon, cucumber etc.; Desbiez and Lecoq, (1997) Plant Pathol. 46:809-829). As in all potyviruses, the ZYMV genome consists of a single messenger-polarity RNA molecule of about 9.6 kb, encapsidated by ˜2000 units of coat protein (CP), forming a helical, flexuous, filamentous particle of about 750 nm long and 11 nm wide (Desbiez, and Lecoq, (1997) Plant Pathol. 46:809-829 and Lisa et al. (1981) Phytopathology 71:667-672).

It is known that conjugation of peptides to carrier proteins restricts conjugation of a peptide to the Potyviral CP. The fact that a potyvirus virion contains ˜2000 copies of this CP makes it an attractive candidate for presentation of a plurality of copies of a heterologous peptide or antigen on the virion surface. Such presentation may be used for purposes such as, for example, vaccination or saturation of peptide binding sites in an organism.

Though there is no high resolution X-ray diffraction data available on the structure of potyvirus CP, there is a considerable amount of information about its topology. Structure predictions together with immunological studies (Desbiez et al. (1997). J. Gen. Virol. 78:2073-2076; Shukla et al. (1988) J. Gen. Virol. 69:1497-1508) of potyvirus CPs demonstrated structural features similar to those documented in tobacco mosaic virus (McLachlan et al. (1980) J. Mol. Biol. 136:203-224) and potato virus X (Sawyer et al. (1987) J. Gen. Virol. 68:1229-1232). Like these proteins potyviral CP is a three-domain protein with variable N- and C-terminal regions exposed on the virion surface and a relatively conserved core domain that interacts with viral RNA (Allison et al. (1985) Virology 147:309-316). ZYMV CP (279 aa) is composed of a 214-216 amino acid core domain flanked by 43-45 and 20 amino acids N- and C-terminal domains respectively, as predicted by Shukla et al., (1989, Adv. Virus Res. 36:273-314). Potyvirus CP putative trypsin protease motif, representing the end of the surface-exposed NT domain, is presumed to be located in one of the Lys and Asp pairs located in the K⁴²DKD motif (Shukla et al. (1988) J. Gen. Virol. 69:1497-1508.). This trypsin cleavage site makes the N-terminal domain of potyvirus an attractive candidate for construction of fusion proteins, because the heterologous portion of the fusion protein may be cleaved enzymatically, either in vitro or in vivo.

Different domains have been associated with distinct functions of CP during viral life cycle. It has been shown that the conserved core, but not the N- and C-terminal is required for virus assembly (Dolja et al. (1995) Virology. 206:1007-1016; Jagadish et al. (1991) J. Gen. Virol. 72:1543-1550; and Varrelmann et al. (2000) J. Gen. Virol. 81:567-576.), plasmodesmatal gating (Rojas et al. (1997) Virology 237:283-295) and cell-to-cell movement (Dolja et al. (1995) Virology. 206:1007-1016). The N-terminal domain has been shown to assist aphid transmission via its DAG motif (Atreya et al. (1991) Proc. Natl. Acad. Sci. USA 88:7887-7891 and Gal-On et al. (1992) J. Gen. Virol. 73:2183-2187), through interaction with the viral encoded helper component-proteinase (HC-Pro; Peng et al., (1998) Journal Gen. Virol. 79:897-904). The prior art teaches that the N-terminal domain of the CP is involved in viral long distance movement and systemic spread as exemplified hereforth. Tobacco etch virus (TEV) mutants with deletions in the CP N- or C-terminal domains produced virions in vivo but the virus was defective in long-distance movement in planta (Dolja et al. (1994) EMBO J. 13:1482-1491 and Dolja et al. (1995) Virology. 206:1007-1016). Also, mutational analysis demonstrated that changes of Ser₄₇ to Pro of the Pea seed borne mosaic potyvirus (PsBMV) CP (Andersen et al. (1998) Virology 241:304-311) and Asp₅ to Lys in the DAG motif of the Tobacco vein mottling potyvirus CP N-terminal domain (Lopez-Moya et al. (1998) J. Gen. Virol. 79:161-165) can modulate the ability of the virus to move systemically in Chenopodium quinoa and tobacco plants respectively. Additionally, substitution of Potato potyvirus A CP Ser₇ to Gly reduced virus accumulation ten fold but did not affect the rate of systemic movement (Andrejeva et al. (1999) J. Gen. Virol. 80:1133-1139). Nevertheless, viral accumulation and long distance movement of Plum pox virus (PPV) were not affected by insertion of 15 and 30 amino acid long non-viral sequence between CP Ala₁₂ and Leu₁₃ (Fernandez-Fernandez et al. (1998) FEBS Lett. 427:229-235). This insertion did not involve a deletion of any part of the PPV authentic CP-NT nor was the heterologous peptide fused to the extreme N-terminus. Thus, it is widely believed that it is infeasible to delete or replace even a portion of the CP N-terminal domain of potyviruses to facilitate the fusion of heterologous peptides. This is because of the apparent indispensability of the CP N-terminal domain of the coat protein for viral viability. Therefore, potyviruses have generally been considered unsuitable as carriers of biologically important peptides attached to the end of the CP N terminal domain or replacing it. Means for attenuating potyviruses in general, and ZYMV in particular, have been described in WO 99/51749 and in Gal-On (2000) Phytopathology 90:467-473). However, the apparent indispensability of the N terminal domain of the coat protein seemed to render these viruses unsuitable as vectors for expressing biologically important peptides on the viral coat.

U.S. Pat. No. 5,958,422 as well as WO9602649 and WO9218618 teach modified plant viruses as vectors for heterologous peptides, including peptides useful for vaccination. However, these patents do not include teachings, which enable use of the N-terminal domain of the potyvirus coat protein. Despite the assertion in the introduction that “The invention can be applied to any plant virus by identifying that part of the virus genome which encodes an exposed portion of a coat protein”, the sole independent claim of this patent is specifically limited to “a foreign peptide insert at a non-terminal site in the coat protein of the virus”. Further, it is well accepted in the prior art (as detailed hereinabove) that there is a requirement for the N-terminus of the CP of potyvirus. Further, Teachings of these patents require harvest of virus particles for vaccination. Such harvest is costly and complex. In contrast, this harvest can be avoided if virus particles are produced in edible plants such as cucurbits.

U.S. Pat. No. 6,034,298 teaches vaccines derived from transgenic plants. The transgenic plants are prepared by transformation with a construct containing a plant promoter and a recombinant antigen. The teachings of this patent lack the advantages of epitope presentation on a viral coat. Further, the teachings of this patent require germ line transformation of the plant. Plants carrying germ line transformations might be subjected to greater scrutiny by regulatory agencies before widespread commercial use is permitted. This scrutiny is a significant disadvantage.

There is thus a widely recognized, previously unmet, need for vectors capable of expressing heterologous peptides at the N-terminus of Potyvirus coat protein, methods for use thereof, plants infected with same and methods of vaccination using same devoid of the above limitations.

### SUMMARY OF THE INVENTION

According to one aspect of the present invention there is provided a recombinant vector for expressing a heterologous peptide at the amino-terminus of a potyvirus coat protein. The vector includes (a) sufficient potyvirus nucleic acid sequence to permit viral replication and spread within a plant infected by the vector; and (b) a heterologous nucleic acid sequence inserted at the amino-terminus of the potyvirus coat protein

According to another aspect of the present invention there is provided a method of transiently expressing at least a portion of a heterologous peptide in at least a portion of a plant. The method includes the steps of: (a) providing a recombinant vector as described hereinabove;

(b) introducing at least one copy of the vector into at least one cell of the plant; and (c) cultivating the plant so that the vector is transcribed, the resulting nucleic acid replicating therein by forming viable potyvirus virions which spread from cell to cell within the plant. In this way, each of the viable potyvirus virions displays on an external surface thereof a plurality of copies of the at least a portion of the heterologous peptide.

According to yet another aspect of the present invention there is provided a plant transiently expressing at least a portion of a heterologous peptide in at least a portion thereof. The plant includes: (a) at least one cell infected with a recombinant vector for expressing the at least a portion of the heterologous peptide, the vector including components as described hereinabove. Within the plant, the vector is transcribed extrachromosomally, thereby forming viable potyvirus virions capable of replicating and spreading from cell to cell within the plant. Further, each of the viable potyvirus virions displays on an external surface thereof a plurality of copies of the at least a portion of a heterologous peptide.

According to still another aspect of the present invention there is provided a method of vaccination. The method includes the steps of: (a) providing a recombinant vector as described hereinabove and the heterologous nucleic acid sequence encoding at least one antigenic determinant; (b) introducing at least one copy of the vector into at least one cell of a plant; (c) cultivating the plant so that the vector is transcribed therein resulting in the formation of viable potyvirus virions which replicate and spread from cell to cell within the plant so that each of the potyvirus virions displays on an external surface thereof a plurality of copies of the at least one antigenic determinant; (d) harvesting at least a portion of the plant; and (e) delivering the potyvirus virions to a subject, such that the plurality of copies of the at least one antigenic determinant contained therein elicit an immune response from the subject.

According to further features in preferred embodiments of the invention described below, the amino-terminus is selected from the group consisting of: (i) an established amino-terminus of a wild type potyvirus coat protein; and (ii) an alternate amino-terminus of a potyvirus coat protein, the alternate amino-terminus arising from an action selected from the group consisting of an insertion, a replacement and a deletion of at least one amino acid residue from the known amino-terminus.

According to still further features in the described preferred embodiments the heterologous nucleic acid sequence encodes at least a portion of the heterologous peptide.

According to still further features in the described preferred embodiments the potyvirus is zucchini yellow mosaic virus (ZYMV).

According to still further features in the described preferred embodiments the potyvirus is selected from the group consisting of ALMV, AmLMV, ArjMV, ArLV, AV-1, BCMV, BCNMV, BYMV, BtMV, BiMoV, CdMV, CVMV, CTLV, CeMV, ChiVMV, CIYVV, CSV, CDV, ComMV, CABMV, CGVBV, DsMV, DSTV, DeMV, GSMV, GEV, GGMV, HVY, HMV, HiMV, IFMV, IMMV, ISMV, JGMV, KMV, LYSV, LMV, MDMV, NDV, NYSV, NoMV, OYDV, ORMV, BRSV, PARMV, PWV, PSBMV, PEMOV, PEPMOV, PESMV, PVMV, PTV, PPV, PKMV, PVA, PVV, PVY, RETBV, SRMV, SMV, SCMV, SPFMV, TAMMV, TEMV, TEV, TVMV, TBBV, TBV, TSBV, TUMV, WMV-2, WVMV, YMV and ZYFV.

According to still further features in the described preferred embodiments the potyvirus nucleic acid sequence and the heterologous nucleic acid sequence are each selected from the group consisting of a DNA sequence, an RNA sequence a cDNA sequence and combinations thereof.

According to still further features in the described preferred embodiments the coat protein of the potyvirus includes an amino-terminal domain.

According to still further features in the described preferred embodiments the amino-terminal domain is modified by deletion of at least one amino acid residue.

According to still further features in the described preferred embodiments fusion to the potyvirus coat protein influences a biological activity of the at least a portion of the heterologous peptide.

According to still further features in the described preferred embodiments the at least a portion of a heterologous peptide is selected from the group consisting of an antigen, a receptor, a ligand, an enzyme, a surfactant, a pore forming molecule, a chaperone, a nucleic acid binding molecule, a transcription factor, an inhibitor, an ion binding molecule, a carbohydrate binding molecule and a signal transducer.

According to still further features in the described preferred embodiments the at least a portion of the heterologous peptide is capable of eliciting an immune response when appropriately administered to an animal.

According to still further features in the described preferred embodiments the animal is selected from the group consisting of an insect, a fish, a bird, a reptile and a mammal.

According to still further features in the described preferred embodiments the mammal is selected from the group consisting of a house pet, a laboratory animal, a sheep, a goat, a cow, a pig, a monkey, an ape and a human being.

According to still further features in the described preferred embodiments the recombinant vector further includes an amino acid substitution in the HC— Pro gene of the conserved FRNK box of the potyvirus nucleic acid sequence, the substitution causing attenuation of the potyvirus.

According to still further features in the described preferred embodiments the recombinant vector further includes an amino acid substitution in the potyvirus nucleic acid sequence, the substitution effectively abolishing aphid transmissibility of the potyvirus.

According to still further features in the described preferred embodiments the heterologous nucleic acid sequence encodes at least a portion of a peptide selected from the group consisting of cMYC, FMDV, His tag and Ovalbumin.

According to still further features in the described preferred embodiments the recombinant vector further includes one additional amino acid residue, the additional amino acid residue facilitating proteolytic excision of the coat protein from within a potyvirus polyprotein.

According to still further features in the described preferred embodiments the additional residue is selected from the group consisting of serine, methionine, glycine, alanine and phenylalanine.

According to still further features in the described preferred embodiments translation of the potyvirus nucleic acid sequence encoding a portion of an amino-terminal domain of a potyvirus coat protein and the heterologous nucleic acid sequence fused thereto produces a fusion protein with an isoelectric point similar to an isoelectric point of a native potyvirus coat protein.

According to still further features in the described preferred embodiments the vector is transcribed extrachromosomally.

According to still further features in the described preferred embodiments the plant is a cucurbit plant.

According to still further features in the described preferred embodiments the method includes the additional step of isolating the potyvirus virions from the at least a portion of the plant.

According to still further features in the described preferred embodiments the step of delivering includes oral administration to the subject.

According to still further features in the described preferred embodiments the step of delivering and the step of harvesting are carried out concurrently.

According to still further features in the described preferred embodiments the step of delivering is accomplished by a means selected from the group consisting of injection, oral administration, intraocular administration, intranasal administration, transdermal delivery, aerosol delivery, intravaginal administration and rectal administration.

According to still further features in the described preferred embodiments the virus vector replicates extrachromosomally.

According to still further features in the described preferred embodiments the virus vector replicates outside a nucleus of the at least one cell of the plant.

According to still further features in the described preferred embodiments heterologous nucleic acid sequence includes at least a portion of at least one member selected from the group consisting of SEQ ID NOs.: 4, 8, 20 and 28.

According to still further features in the described preferred embodiments the heterologous nucleic acid sequence encodes at least a portion of at least one peptide selected from the group consisting of SEQ ID NOs.: 5, 9, 21 and 29.

According to still further features in the described preferred embodiments the vector includes at least a portion of at least one item selected from the group consisting of SEQ ID Nos.: 6, 7, 10-19, 22-27, 30 and 31.

The present invention successfully addresses the shortcomings of the presently known configurations by providing vectors for expressing heterologous peptides at the amino-terminus of Potyvirus Coat Protein, methods for use thereof, plants infected with same and methods of vaccination using same. It is an important advantage of the present invention that no germ line transformation of a plant is required in order to express the heterologous peptide.

### BRIEF DESCRIPTION OF THE DRAWINGS

The invention is herein described, by way of example only, with reference to the accompanying drawings, figures and tables. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C illustrate characterization of AGII-His and AGII-HisΔ8 in systemically infected squash leaves;

FIGS. 2A and B demonstrate that the His-tag is exposed on the surface of AGII-His and AGII-HisΔ8 virions;

FIGS. 3A-C illustrate characterization of AGII-Myc in systemically infected squash leaves;

FIGS. 4A-C illustrate characterization of Myc-tagged AGII deletion mutants in systemically infected squash leaves;

FIGS. 5A and B illustrate that the Myc-tag is exposed on the surface of chimeric virions;

FIGS. 6A-C illustrate characterization of FMDV-tagged AGII mutants in systemically infected squash leaves; and

FIGS. 7A-C demonstrate induction of anti ZYMV CP immune response in mice.

### DESCRIPTION OF THE PREFERRED EMBODIMENTS

The present invention is of vectors for expressing heterologous peptides at the amino-terminus of Potyvirus Coat Protein (CP), methods for use thereof, plants infected with same and methods of vaccination using same.

Specifically, the present invention can be used to express at least a portion of a heterologous peptide on the surface of potyvirus virions so that a portion of a plant, for example a cucurbit fruit, infected therewith may be used as a source of material for vaccination, pharmaceutical or diagnostic applications. Zucchini Yellow Mosaic Potyvirus (ZYMV) is especially well suited for use in the present invention, although the scope of the claimed invention includes the use of other potyvirus species.

The principles and operation of vectors for expressing heterologous peptides at the amino-terminus of Potyvirus Coat Protein (CP), methods for use thereof, plants infected with same and methods of vaccination using same according to the present invention may be better understood with reference to the figures and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is embodied by a recombinant vector for expressing a heterologous peptide at the amino-terminus of a potyvirus coat protein. The vector includes sufficient potyvirus nucleic acid sequence to permit viral replication and spread within a plant infected by the vector. The vector further includes a heterologous nucleic acid sequence inserted at the amino-terminus of the potyvirus coat protein. The heterologous nucleic acid sequence may encode, for example, at least a portion of a peptide such as cMYC, FMDV, His tag or Ovalbumin.

According to various preferred embodiments of the invention, the amino-terminus may be, for example, an established amino-terminus of a wild type potyvirus coat protein or an alternate amino-terminus of a potyvirus coat protein. The alternate amino-terminus may arise, for example, from an insertion, a replacement or a deletion of at least one amino acid residue from the known amino-terminus. The heterologous nucleic acid sequence preferably encodes at least a portion of the heterologous peptide.

Preferably, the potyvirus is zucchini yellow mosaic virus (ZYMV). Alternately, the potyvirus may be, for example, ALMV, AmLMV, ArjMV, ArLV, AV-1, BCMV, BCNMV, BYMV, BtMV, BiMoV, CdMV, CVMV, CTLV, CeMV, ChiVMV, CIYVV, CSV, CDV, ComMV, CABMV, CGVBV, DsMV, DSTV, DeMV, GSMV, GEV, GGMV, HVY, HMV, HiMV, IFMV, IMMV, ISMV, JGMV, KMV, LYSV, LMV, MDMV, NDV, NYSV, NoMV, OYDV, OrMV, BRSV, ParMV, PWV, PSbMV, PeMoV, PepMoV, PeSMV, PVMV, PTV, PPV, PkMV, PVA, PVV, PVY, ReTBV, SrMV, SMV, SCMV, SPFMV, TamMV, TeMV, TEV, TVMV, TBBV, TBV, TSBV, TuMV, WMV-2, WVMV, YMV or ZYFV.

According to preferred embodiments of the invention, the potyvirus nucleic acid sequence and the heterologous nucleic acid sequence may each include a DNA sequence, an RNA sequence a cDNA sequence or combinations thereof.

According to still further features in the described preferred embodiments the coat protein of the potyvirus includes an amino-terminal domain. In some cases, amino-terminal domain is modified by deletion of at least one amino acid residue. This deletion is typically from the amino terminus of the amino terminal domain.

Preferably, fusion to the potyvirus coat protein influences a biological activity of the at least a portion of the heterologous peptide. The at least a portion of a heterologous peptide may include, but is not limited to, an antigen, a receptor, a ligand, an enzyme, a surfactant, a pore forming molecule, a chaperone, a nucleic acid binding molecule, a transcription factor, an inhibitor, an ion binding molecule, a carbohydrate binding molecule or a signal transducer.

According to some preferred embodiments, the at least a portion of the heterologous peptide is capable of eliciting an immune response when appropriately administered to an animal. The animal may be, for example, an insect, a fish, a bird, a reptile or a mammal. The mammal may be, for example, a house pet, a laboratory animal, a sheep, a goat, a cow, a pig, a monkey, an ape or a human being.

The recombinant vector may further include an amino acid substitution in the HC— Pro gene of the conserved FRNK box of the potyvirus nucleic acid sequence. This substitution causes attenuation of the potyvirus. One ordinarily skilled in the art will be able to make such a substitution, for example, as described in Gal-On (2000) Phytopathology 90:467-473.

Alternately, or additionally, the recombinant vector may include an amino acid substitution in the potyvirus nucleic acid sequence, which effectively abolishes aphid transmissibility of the potyvirus. One ordinarily skilled in the art will be able to make such a substitution, for example, as described in Gal-On et al. (1992) J. Gen. Virol. 73:2183-2187.

Preferably, the recombinant vector further includes one additional amino acid residue, which facilitates proteolytic excision of the coat protein from within a potyvirus polyprotein. This additional residue may be, for example serine, methionine, glycine, alanine or phenylalanine.

Preferably, translation of the potyvirus nucleic acid sequence encoding a portion of an amino-terminal domain of a potyvirus coat protein and the heterologous nucleic acid sequence fused thereto produces a fusion protein with an isoelectric point similar to an isoelectric point of a native potyvirus coat protein.

It will be appreciated that the vector, since it encodes a potyvirus, is transcribed extrachromosomally within cells of a plant infected therewith. Preferably, the vector is employed to infect a plant such as a cucurbit plant. As a result, it is possible to produce edible fruits, which contain seeds, which have not undergone a germ line transformation, despite the fact that the fruit itself contains a significant amount of a heterologous peptide.

As non-limiting examples of embodiments of the invention, the heterologous nucleic acid sequence may include at least a portion of SEQ ID NO.: 4, 8, 20 or 28 or combinations thereof. According these exemplary embodiments the heterologous nucleic acid sequence encodes at least a portion of a peptide designated by SEQ ID NOs.: 5, 9, 21 or 29 or combinations thereof.

The recombinant vector of the present invention may include, but is not limited to, a sequence such as those delimited in SEQ ID NOs.: 6, 7, 10-19, 22-27, 30 and 31 or a portion thereof.

The invention is further embodied by a method of transiently expressing at least a portion of a heterologous peptide in at least a portion of a plant. The method includes the step of providing a recombinant vector as described hereinabove. The method further includes the step of introducing at least one copy of the vector into at least one cell of the plant. The method further includes the step of cultivating the plant so that the vector is transcribed. The nucleic acid resulting from this transcription replicates within the at least one cell of the plant by forming viable potyvirus virions. These virions spread from cell to cell within the plant. In this way, each of the viable potyvirus virions displays on an external surface thereof a plurality of copies of the at least a portion of the heterologous peptide. The method is well suited to use with cucurbit plants.

The invention is further embodied by a plant transiently expressing at least a portion of a heterologous peptide in at least a portion thereof. The plant includes at least one cell infected with a recombinant vector for expressing the at least a portion of the heterologous peptide. The vector includes components as described hereinabove. Within the plant, the vector is transcribed extrachromosomally, thereby forming viable potyvirus virions capable of replicating and spreading from cell to cell within the plant. Further, each of the viable potyvirus virions displays on an external surface thereof a plurality of copies of the at least a portion of a heterologous peptide.

According to still another aspect of the present invention there is provided a method of vaccination. The method includes the step of providing a recombinant vector as described hereinabove. Vectors used in conjunction with this embodiment of the invention contain a heterologous nucleic acid sequence, which encodes at least one antigenic determinant. The method further includes the steps of introducing at least one copy of the vector into at least one cell of a plant and cultivating the plant so that the vector is transcribed therein resulting in the formation of viable potyvirus virions which replicate and spread from cell to cell within the plant so that each of the potyvirus virions displays on an external surface thereof a plurality of copies of the at least one antigenic determinant. The method further includes the steps of harvesting at least a portion of the plant and delivering the potyvirus virions to a subject. Delivery is performed so that the plurality of copies of the at least one antigenic determinant contained therein elicit an immune response from the subject. The method may include the additional step of isolating the potyvirus virions from the at least a portion of the plant.

The step of delivering may be accomplished by means including, but not limited to, injection, oral administration, intraocular administration, intranasal administration, transdermal delivery, aerosol delivery, intravaginal administration and rectal administration. Preferably, the step of delivering includes oral administration to the subject. Still more preferably, the step of delivering and the step of harvesting are carried out concurrently.

The vector replicates extrachromosomally, preferably outside a nucleus of the at least one cell of the plant.

In the Figures:

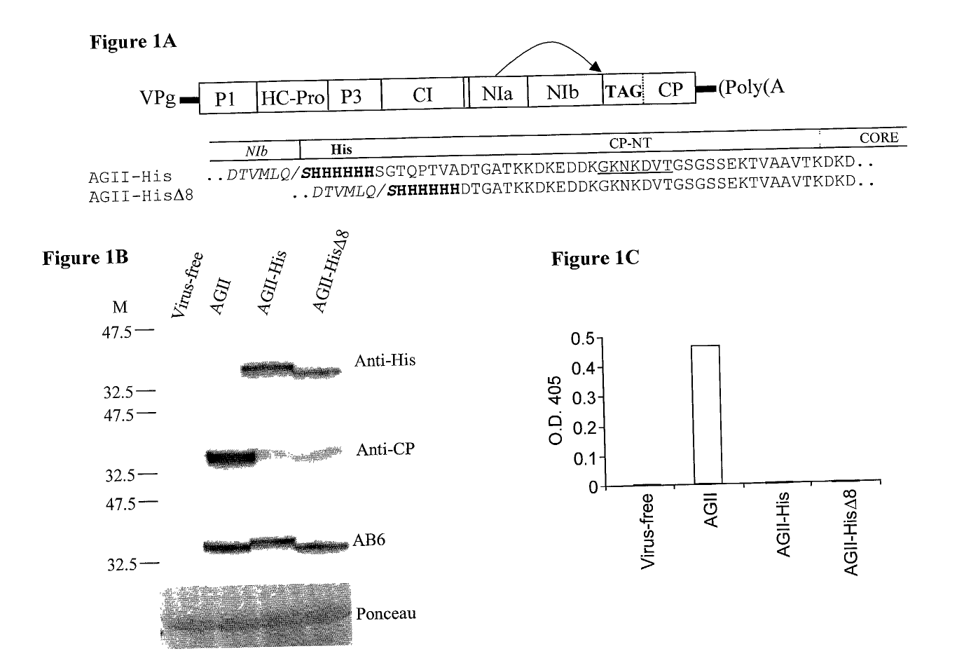

FIGS. 1A-C illustrate characterization of AGII-His and AGII-HisΔ8 in systemically infected squash leaves. FIG. 1A is a schematic representation of the AGII-His and AGII-HisΔ8 CP-NT region. The insertion site of the His peptide (TAG) in the genomic map of the AGII virus and the amino acid sequences at this site are shown. Partial ORFs of NIb and CP are graphically indicated by a rectangle separated by a solid vertical line. A dotted vertical line separates the His-tag (His), CP N-terminus (CP-NT) and CP core (CORE) ORFs from one another. Residues encoding the NIa protease motif are shown in italics. The amino acid residues recognized by AB6 monoclonal antibody are underlined.

FIG. 1B shows results of an immunoblot analysis of AGII-His and AGII-HisΔ8. Total extracts (15 μl) of systemically infected squash leaves were analyzed on 12.5% SDS-PAGE, blotted and probed with the indicated antibody. Extracts from virus-free plants (Virus-free) were used as a negative control. All samples, including virus-free, were collected from developmentally equivalent leaves at 21 dpi (days post infection). Relative loading of protein in each lane is shown by Ponceau staining. The positions of molecular-mass standards (kDa) are indicated on the left.

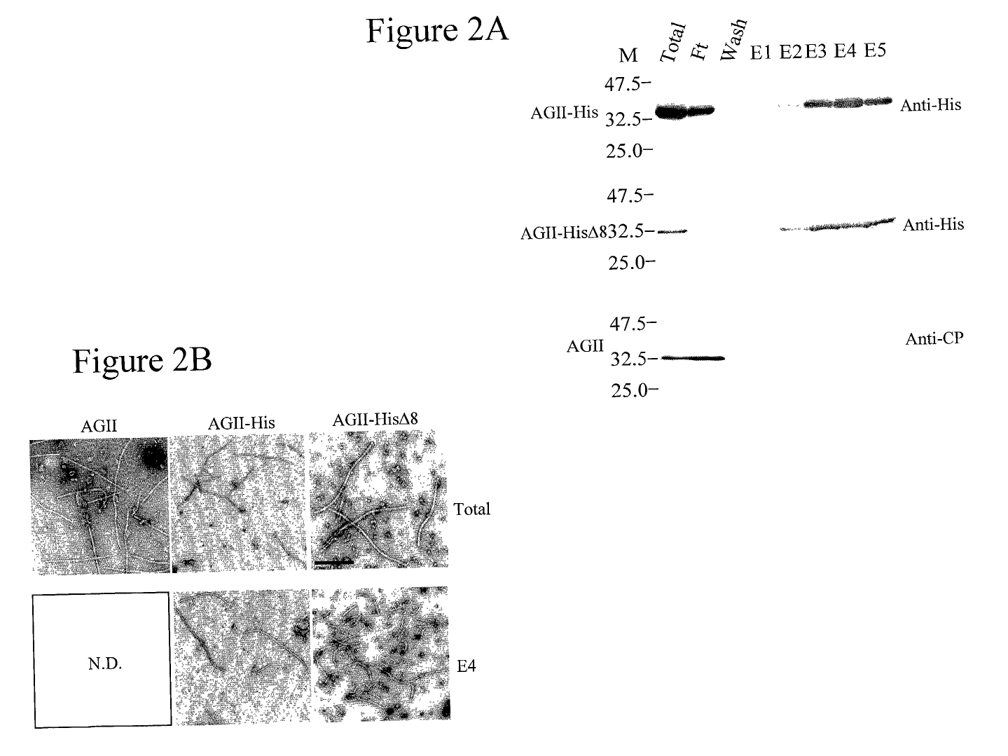

FIG. 1C shows results of DAS-ELISA analysis with anti-CP antibody as described hereinabove. Samples are similar to those employed in FIG. 1B. Each result is the average of three independent samples taken from three different plants FIGS. 2A and B demonstrate that tag-tag is exposed on the surface of AGII-His and AGII-HisΔ8 virions.

FIG. 2A illustrates one-step affinity purification of His-tagged virions on Ni²⁺ charged resin. Extracts from AGII, AGII-His and AGII-His? 8 systemically infected squash leaves were mixed with Ni²⁺-charged resin and subjected to Ni²⁺-affinity chromatography. Equal volumes of pre-Ni²⁺ mixing fraction (Total), column effluent fraction (Ft), final 75 mM imidazole wash fraction (Wash) and the first to fifth 300 mM imidazole eluted fractions (E1-E5, respectively) were analyzed by immunoblot with the indicated antibody. The positions of molecular-mass standards (kDa) are indicated on the left.

FIG. 2B presents transmission electron micrographs of AGII, AGII-His and AGII-His? 8 virions from either the Total or E4 fractions described in FIG. 2A. N.D. indicates not detected. The reference bar on the top right micrograph indicates 430 nm.

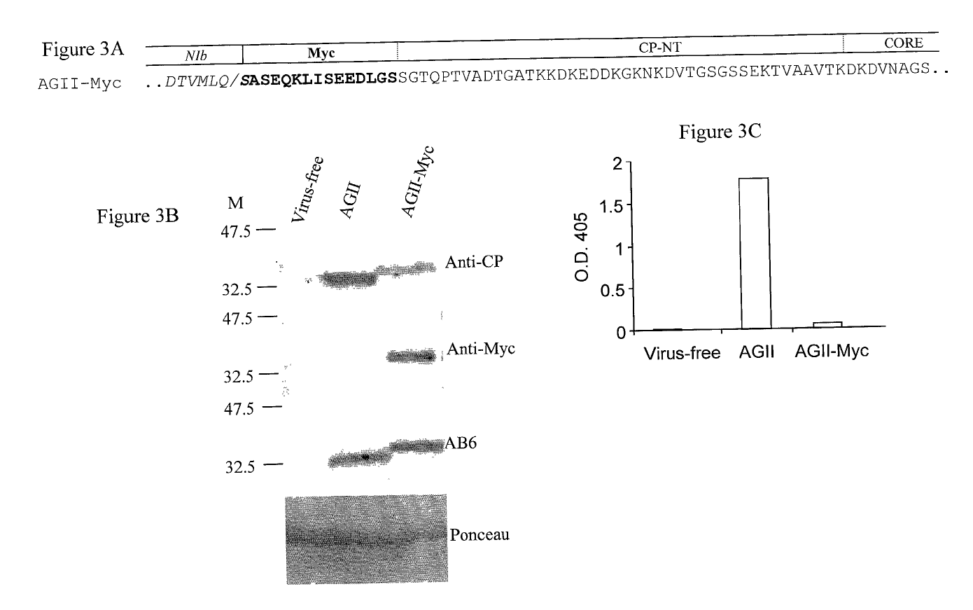

FIGS. 3A-C illustrate characterization of AGII-Myc in systemically infected squash leaves FIG. 3A is a schematic presentation of the AGII-Myc CP-NT region. Partial open reading frames (ORF) of NIb and CP are indicated by a rectangle separated by a solid vertical line. The dotted vertical lines separate the Myc-tag (Myc), CP N-terminus (CP-NT) and CP core (CORE) domains from each other. Residues encoding the NIa protease motif are shown in italics. Residues encoding the Myc and shown in bold.

FIG. 3B presents results of an immunoblot analysis of AGII-Myc. Total extracts (15 μl) of systemically infected squash leaves were analyzed on 12.5% SDS-PAGE, blotted and probed with the indicated antibody. Extracts from virus-free squash plants (Virus-free) were used as a negative control. All samples, including Virus-free, were collected from developmentally equivalent leaves at 21 dpi. Relative loading of protein in each lane is shown by Ponceau staining. The positions of molecular-mass standards (kDa) are indicated on the left;

FIG. 3C summarizes results of a DAS-ELISA analysis with anti-CP of samples as described for FIG. 3B. Each result is the average of three independent samples taken from three different plants.

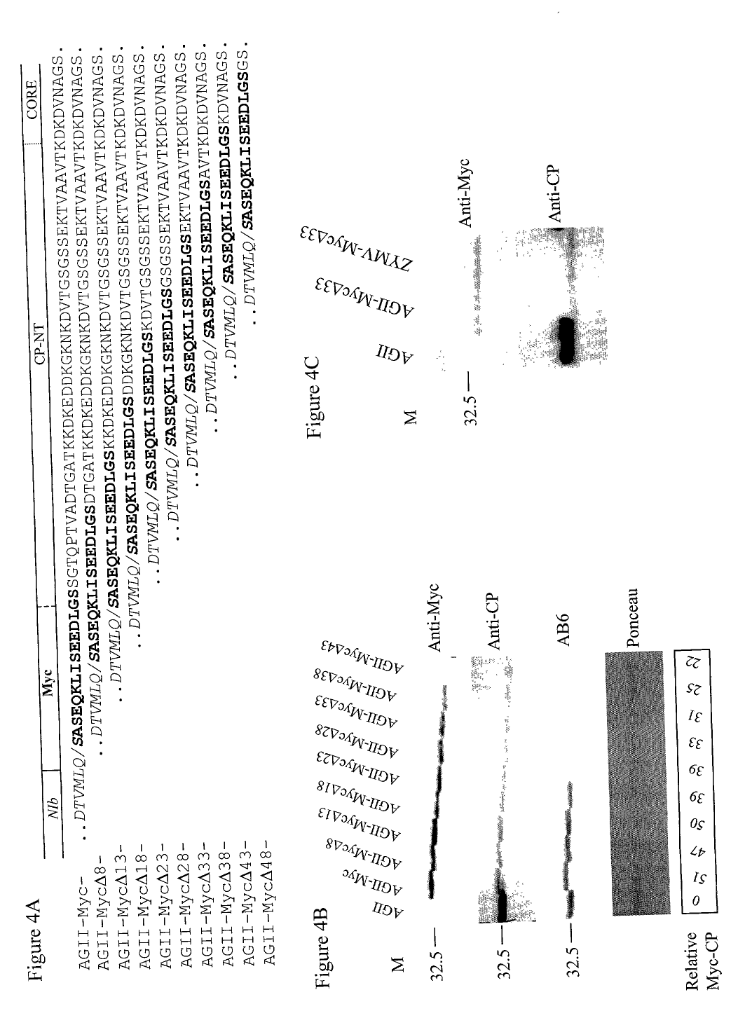

FIG. 4 is a characterization of Myc-tagged AGII deletion mutants in systemically infected squash leaves.

FIG. 4A is a schematic presentation of the CP-NT region of Myc-tagged AGII deletion mutants. Partial ORFs of NIb and CP are graphically indicated by a rectangle separated by a solid vertical line. A dotted vertical line separates the Myc-tag (Myc), CP N-terminus (CP-NT) and CP core (CORE) domains from each other. Residues encoding the NIa protease motif and Myc are shown in italics and bold respectively.

FIG. 4B presents results of an immunoblot analysis of Myc-Tagged AGII deletion mutants. Total extracts (10 μl) of systemically infected squash leaves were analyzed on 12.5% SDS-PAGE, blotted and probed with indicated antibody. All samples were collected from developmentally equivalent leaves at 21 dpi. Relative loading of protein in each lane is shown by Ponceau staining. The positions of molecular-mass standards (kDa) are indicated on the left. Relative amounts of Myc-CP fusion protein were determined by a densitometric scan of the anti-Myc signal and are shown at the bottom of the figure.

FIG. 4C presents results of an immunoblot analysis of AGII, AGII-MycΔ33 and ZYMV-MycΔ33. Total extracts (15 μl) of systemically infected squash leaves were analyzed on 12.5% SDS-PAGE, blotted and probed with indicated antibody. All samples were collected from developmentally equivalent leaves at 21 dpi. The positions of molecular-mass standards (kDa) are indicated on the left.

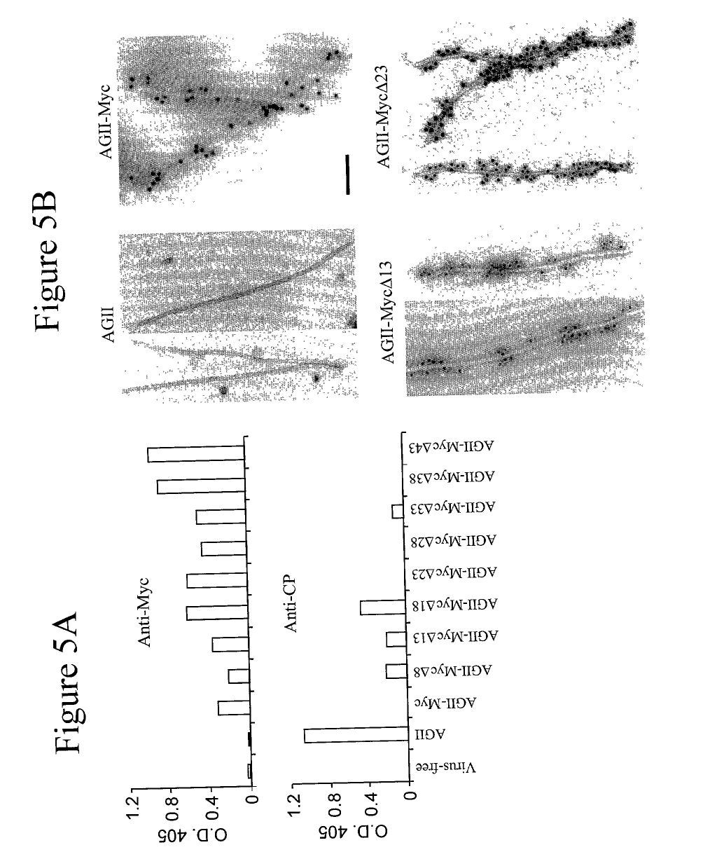

FIGS. 5A and B illustrate that the Myc-tag is exposed on the surface of chimeric virions.

FIG. 5A illustrates comparative detection of CP and Myc epitopes on AGII and Myc-tagged deletion mutant viruses. The relative amount of each virion was determined by DAS-ELISA with the indicated antibody and is the average of three independent samples taken from three different plants 21 dpi.

FIG. 5B illustrates results of immunogold labeling of AGII, AGII-Myc, AGII-MycΔ13 and AGII-MycΔ23. Partially purified AGII, AGII-Myc, AGII-MycΔ13 and AGII-MycΔ23 virions were incubated with anti-Myc monoclonal antibody. The micrographs are each a composite from several fields of view. The reference bar on the top right micrograph indicates 15n0 n.

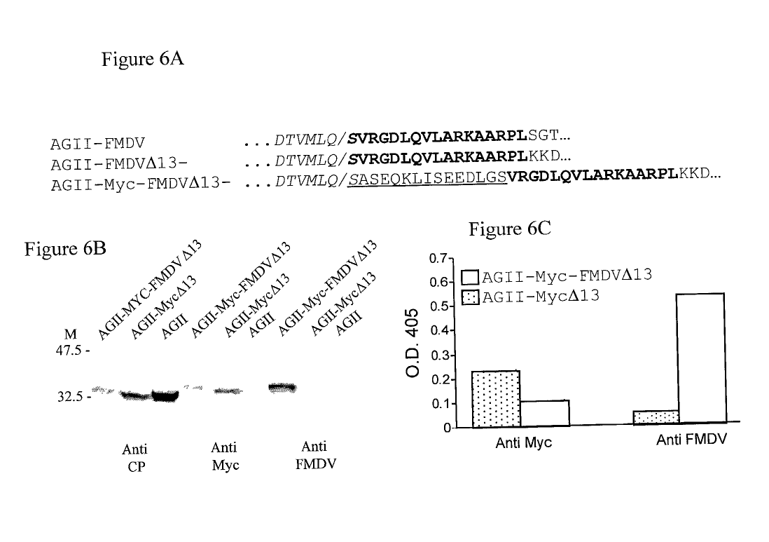

FIGS. 6A-C illustrate characterization of FMDV-tagged AGII mutants in systemically infected squash leaves.

FIG. 6A presents the amino acid sequences of FMDV-Tagged AGII mutants at the NIb/CP insertion point. Residues encoding FMDV and Myc tags are shown in bold and underlined respectively. Residues encoding NIa protease motif are shown in italics.

FIG. 6B presents the results of an immunoblot analysis of AGII, AGII-MycΔ13 and AGII-Myc-FMDVΔ13. Total extracts of systemically infected squash leaves were analyzed on 12.5% SDS-PAGE, blotted and probed with the indicated antibody. All samples were collected from developmentally equivalent leaves at 21 dpi. The positions of molecular-mass standards (kDa) are indicated on the left.

FIG. 6C summarizes the results of a DAS-ELISA analysis of AGII-MycΔ13, and AGII-Myc-FMDVΔ13 samples as described for FIG. 6B. Analysis was performed with anti-Myc or anti-FMDV antibody in two different ELISA plates as indicated. Each result is the average of three independent samples taken from three different plants.

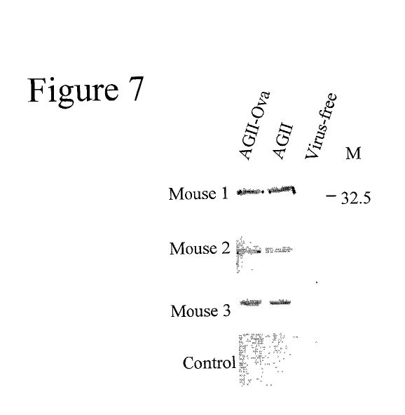

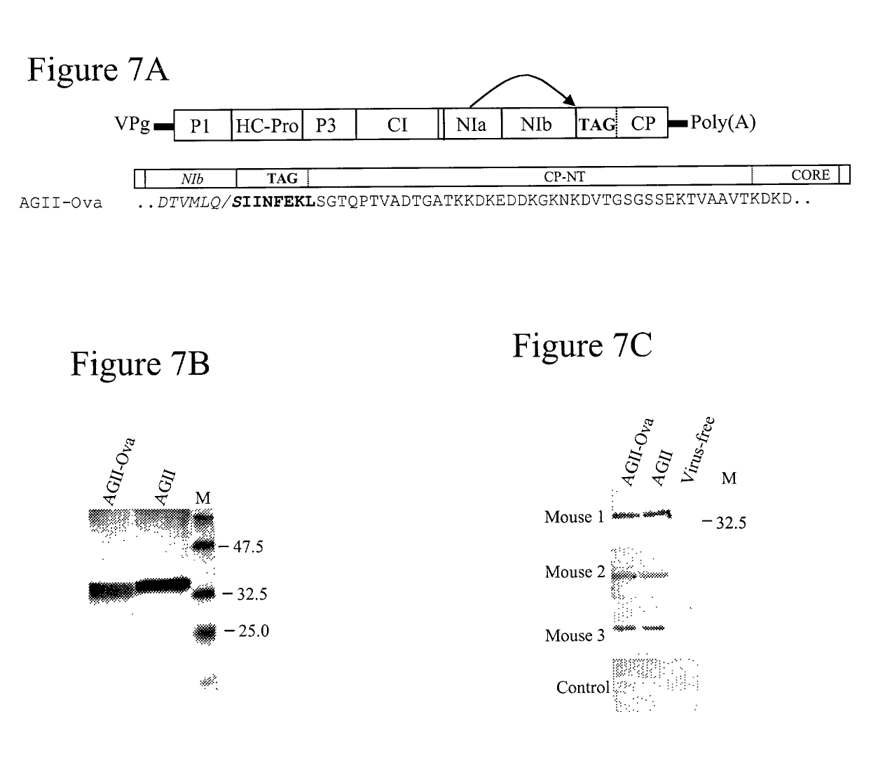

FIGS. 7A-C demonstrate induction of an anti-ZYMV CP immune response in mice.

FIG. 7A is a schematic presentation of the AGII-Ova CP-NT region. Partial ORFs of NIb and CP are indicated by a rectangle separated by a solid vertical line. A dotted vertical line separates the Ova-tag (Ova), CP N-terminus (CP-NT) and CP core (CORE) domains from each other. Residues encoding the NIa protease motif and Ova are shown in italics and bold respectively;

FIG. 7B presents results of an immunoblot analysis of AGII-Ova. Total extracts (10 μl) of systemically infected squash leaves were analyzed on 12.5% SDS-PAGE, blotted and probed with the indicated antibody. All samples were collected from systemically infected leaves at 21 dpi. The positions of molecular-mass standards (kDa) are indicated on the right.

FIG. 7C presents results of an immunoblot analysis conducted using serum taken from AGII-Ova orally and intra-nasally vaccinated mice as described hereinabove. Total extracts (10 μl) of healthy squash leaves (Virus-free) or 0.5 μg partially purified CP or CP-Ova proteins, were separated on 12.5% SDS-PAGE, blotted and probed with the indicated serum (1:500 dilution) of vaccinated mice 1, 2, or 3 (as indicated) or control mice. The positions of molecular-mass standards (kDa) are indicated on the right.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes 1-111 Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); and “Using Antibodies: A Laboratory Manual” (Ed Harlow, David Lane eds., Cold Spring Harbor Laboratory Press (1999)) all of which are incorporated by reference as if fully set forth herein. Other general references are provided throughout this document. The procedures therein are believed to be well known in the art and are provided for the convenience of the reader. All the information contained therein is incorporated herein by reference.

For purposes of this specification and the accompanying claims, the term “dpi” is an abbreviation of the phrase “days post inoculation”.

The following methods were employed in performing experiments described in the examples recited hereinbelow:

Construction of virus mutants: Constructs containing various CP fusions including at least a portion of a foreign peptide were created by PCR using CP of the AGII strain of ZYMV potyvirus (SEQ ID NO: 1) as a template. Sense primers contained a PstI site at their 5′ end followed by the indicated sequence tag, and a homologous CP sequence with or without deletion. The CP homologous antisense primer contained an MluI site. The amplified fragments were double digested by PstI and MluI and cloned into the partial clone pKS?SacI-PstI-poly (Arazi et al., 2001, Journal of Virology 75:6329-6336). pKS?SacI-PstI-poly clones were double-digested by SacI/MluI, and the resulting fragments containing tags were cloned into the AGII genome to create AGII-tagged mutants.

CP-NT deletions without a sequence tag fusion. Specifically:

AGII? 8 includes (SEQ ID NO: 26) encoding (SEQ ID NO.: 2; Amino acids: 9-279);

AGII ? 13 includes (SEQ ID NO: 27) encoding (SEQ ID NO.: 2; Amino acids: 14-279); and

AGII ? 33 includes (SEQ ID NO: 25) encoding (SEQ ID NO.: 2; Amino acids: 34-279:

These serial deletions were constructed by the same strategy mentioned above using sense primers flanked by a PstI site at their 5′ end followed by a homologous CP deleted sequence.

In summary, AGII? 8; AGII? 13 and AGII? 33 correspond to SEQ ID NO.: 1 with 8, 13 and 33 bases respectively deleted from the 3′ end thereof.

The AGII-Myc-FMDV? 13 (SEQ ID NO.: 24) construct was generated by the same strategy described hereinabove using a sense primer flanked by a BamHI site at its 5′ end followed by FMDV sequence tag and a homologous CP deleted (? 13) sequence starting from nucleotide 8580 (Arazi et al., 2001, Journal of Virology 75:6329-6336). The amplified PCR fragments were double digested by BamHI and MluI and cloned into a partial clone pKS?SacI-PstI-poly already containing a Myc-tagged CP digested by BamHI (underlined) located in the 3′ of the Myc tag (SEQ ID NO.: 8) and MluI.

Portions of Foreign Peptides:

TABLE 1
Portions of foreign peptides fused to the N-terminus of the
ZYMV-AGII CP.

| | | | SEQ ID | |
|---|---|---|---|---|
| | | | NOs | |
| | | | Nucleic | |
| | Length | Sequence | acid; | |
| peptide | in BP | 5′ to 3′ | amino acid | reference |
| His tag | 21 | TCACACCATC | 4; 5 | |
| | | ACCATCACCA | | |
| | | T | | |
| Myc tag | 45 | TCAGCATCAG | 8; 9 | Evan et al.( 1985) Mol. Cell |
| | | AGCAGAAGCT | | Biol. 12:3610-3616 |
| | | CATTTCAGAG | | |
| | | GAGGATCTCG | | |
| | | GATCC- | | |
| FMDV | 51 | AGTGTGAGAG | 20; 21 | Strohmaier et al. (1982) J. Gen. |
| | | GAGATCTTCA | | Virol. 59:295-306. |
| | | AGTGCTTGCA | | |
| | | CGAAAAGCAG | | |
| | | CAAGACCACT | | |
| | | T | | |
| Ovalbumin | 56 | CAGCTGCAGT | 28; 29 | Ovalbumin residues 257-264; |
| | | CCATTATTAA | | (Porgador A, et al. (1997) J |
| | | TTTCGAAAAG | | Immunol. 158, 834-41) |
| | | TTGTCAGGCA | | |
| | | CTCAGCCAAC | | |
| | | TGTGGC | | |

Plant growth, inoculation and symptom evaluation: Squash (Curcurbita pepo L. cv. Ma'ayan), cucumber (Cucumis sativus L. cv. Shimshon) and melon (Cucumis melo L. cv. Arava) plants were grown in a growth chamber under continuous light at 23° C. Seedlings were selected for experimental use when their cotyledons were fully expanded. Particle bombardment inoculation was performed with a hand-held device using published methods (Gal-On et al. (1997) J. Virol. Meth. 64:103-1l0). Mild virus symptoms would be observable only in squash, as the AGII virus is symptomless on other cucurbits, therefore, squash was chosen for testing the systemic infectivity of various viral constructs. After bombardment or mechanical inoculation, squash seedlings were grown and examined daily for symptom development, and the first appearance of symptoms on non-inoculated leaves was recorded.

RT-PCR analysis of recombinant virus progeny: RT-PCR of viral progeny was conducted in a one-tube single-step method modified from Sellner et al., (28) in 50 microliter volume with the CP-NT flanking primers (SEQ ID NO.: 32) 5′-AGCTCCATACATAGCTGAGACA-3′ and (SEQ ID NO.: 33) 5′-TGGTTGAACCAAGAGGCGAA-3′ in a mixture containing 1.5 mM MgCl₂; 125 μM dNTPs; 1× Sellner buffer: [10× Sellner buffer contains: 670 mM Tris-HCl; 170 mM (NH₄)₂SO₄; 10 mM P-mercapto-ethanol; 2 mg/ml gelatin (Aldrich, calf skin 225 bloom); 60 μM EDTA pH 8.0 (Sellner et al., 1992)]; 100 ng of each specific primer; 2 units of Taq polymerase; 5 units of AMV-RT (Chimerex USA); 2-5 μg total RNA. RT-PCR cycles were as follows: 46° C. 30 min; 94° C. 2 min, followed by 33 cycles at 94° C., 60° C. and 72° C., each of 30 s., and a final cycle of 5 min at 72° C. Resulting amplified fragments were directly sequenced with a homologous nested primer 5′-CATTTCCTTTCACGCGTGGC-3′ (SEQ ID NO.: 3).

Total protein extraction of systemically infected squash leaves: Three independent squash seedlings were inoculated by particle bombardment with each of the various cDNA constructs. Leaf sample (70 mg; 6 leaf disks, 2 of each plant) was collected in microcentrifuge tubes from symptom expressing leaves 14 or 21 days post inoculation (dpi). Sample was ground in 150 microliters of ESB buffer (75 mM Tris-HCl pH 6.8, 9 M Urea, 4.5% (v/v) SDS, 7.5% (v/v) beta-mercaptoethanol), boiled for 5 min and cooled on ice. Cooled homogenates were centrifuged for 10 min at 10,000×g and 100 microliters of the supernatant containing total leaf proteins was collected and mixed with 100 microliters of 2× protein sample buffer. 10-15 microliters of the mixture was subjected to SDS-PAGE and immunoblot.

DAS-ELISA: Elisa assays were performed essentially as previously published (Gal-On (2000) Phytopathology 90:467-473. ). Briefly, infected plant material (105 mg, 9 leaf disks, 3 of each plant) was ground in ELISA sample buffer and centrifuged for 10 min at 10,000 ×g. Supernatant (100 microliters of each sample) was loaded on an ELISA plate coated with antiserum against ZYMV-CP (1:2000). DAS-ELISA was performed according to (15) with either anti-CP alkaline phosphatase conjugate (1:2000), anti-FMDV polyclonal antibody (1:2000) followed by anti-rabbit alkaline phosphatase conjugate (1:4000) or anti-Myc (1:2500) monoclonal antibody followed by anti-mouse alkaline phosphatase conjugate (1:4000).

Affinity purification of His tagged AGII virions with Ni²⁺-charged resin and electron microscopy observation: Squash seedlings were inoculated with constructs containing cDNAs for CP of AGII (SEQ ID NO.: 1), AGII-His (SEQ ID NO.: 6) and AGII-HisΔ8 (SEQ ID NO.: 7). Samples of leaves (2 g) were taken 11 dpi, and homogenized by mortar and pestle in 6 ml of chilled HB (0.1 M borate pH 8.0 and 20 mM imidazole). The homogenate was filtered through one layer of Miracloth (Calbiochem, USA) and the resultant filtrate centrifuged at 2000×g for 10 min at 4° C. The supernatant (designated Total) was collected and mixed with 600 microliters of a 50% slurry of Ni²⁺-charged resin (Cytosignal, USA) that had been equilibrated with HB. The mixture was stirred for 3 hr at 4° C. and loaded on an empty column (BioRad, USA). Gravity flow though (Ft) was collected. The column then washed with 20-column volumes of HB and 10-column volumes of wash buffer (0.1 M borate pH 8.0 and 50 mM imidazole) to eliminate proteins non-specifically bind Ni²⁺. Bound virions were eluted by sequential additions of half column volume of elution buffer (0.1 M borate pH 8.0 and 300 mM imidazole). Samples from Total and fourth eluted fraction were mounted on formvar carbon-coated grids. The grids were negatively stained with 2% uranyl acetate for two minutes. Micrographs were obtained with a JEOL JEM-100CXII electron microscope.

Virus partial purification and immunogold labeling: Infected leaf material was collected 21 dpi and ground with 2 w/v of borate buffer (borate 0.5 M pH. 8.0, 1 mM EDTA), half w/v chloroform and half w/v CCl₄. Extract was centrifuged at 10,000×g for 15 min at 4° C. The supernatant was collected and filtered through three layers of Miracloth (Calbiochem, USA) and the resultant filtrate was loaded on a 20% sucrose cushion in borate buffer (3/1 v/v). Partially purified virions were pelleted by ultra centrifugation at 140,000×g for two and half hours at 4° C. Pellet was dissolved by shaking over night with 1 ml (per tube) of 1/10 diluted borate buffer at 4° C. Ten microliters of partially purified AGII (SEQ ID Nos.: 1 and 2 ), AGII-Myc (SEQ ID NO.: 10), AGII-MycΔ13 (SEQ ID NO.: 12) and AGII-MycΔ33 (SEQ ID No.: 16) were adsorbed onto formvar grids for two min. After a 10 min washing step with TBG (20 mM Tris-HCl pH 8.2, 225 mM NaCl, 1% calf skin gelatin (Sigma, USA), 0.1% bovine serum albumin) samples were incubated with an anti-Myc monoclonal antibody (Sigma, USA) for 15 min. After six washing steps with TBG buffer, samples were incubated for a further 15 min with a 10 nm gold-labeled anti-mouse IgG. Grids were finally washed six times with TBG buffer and three times with filtered DDW before staining with 2% uranyl acetate for two minutes. Micrographs were obtained with a JEOL JEM-100CXII electron microscope.

Antibodies:

The Anti His—A monoclonal antibody referred to herein is an anti poly-histidine antibody obtained from Sigma Chemical Co., U.S.A. (product No. H-1029).

The Anti Myc—A monoclonal antibody referred to herein is an anti human c-myc antibody clone 9E10, (Sigma, product No. M-5546).

The Anti FMDV—Polyclonal antibody referred to herein is an antibody against the FMDV coat protein synthetic peptide kindly provided by Dr. Y. Strum of the Kimron Veterinary Institute, Beit Dagan, Israel.

AB6—A monoclonal antibody against a specific heptapeptide (GKNKDVT) in ZYMV CP-NT (Desbiez et al., 1997, Journal of General Virology).

Anti CP—Polyclonal antibody against the ZYMV coat protein (Antignus et al. (1988) Phytoparasitica 17:289-298). Oral and intranasal application of chimeric AGII-Ova virion to mice:

AGII (SEQ ID Nos.: 1 and 2) and AGII-Ova (SEQ ID NO.: 30) virions were partially purified from squash leaves as described hereinabove and resuspended in 1/10 diluted borate buffer to a concentration of 5.8 μg/μl. Forty μl of AGII-Ova (SEQ ID NO.: 30) virions were given orally (20 μl) and intra-nasally (20 μl ) to mice. Borate buffer was administrated similarly to control mice. On day 16 mice were given an additional 20 μl of AGII-Ova (SEQ ID NO.: 30) intra-nasally. Serum was taken 51 days post first immunization.

### Example 1

### Examination of the ability of AGII containing a foreign peptide fused to the N-terminus of the coat protein to accumulate and systemically infect an inoculated plant

In order to evaluate the AGII (SEQ ID Nos.: 1 and 2) potyvirus as an epitope presentation system, a 21-nucleotide sequence encoding a seven-residue peptide (six histidines with a serine residue at its N-terminus' (SEQ ID NOs.: 4 and 5) was cloned into the AGII genome either with or without N-terminal deletions of the CP.

The serine residue was added to the histidine tag in order to enable processing of the potyvirus polyprotein by the NIa protease (Riechmann, J. L., et al. (1992). J. Gen. Virol. 73:1-16 and FIG. 1A). This created a translational fusion of the cloned sequence with either a full-length CP (AGII-His; FIG. 1A; SEQ ID NO.: 6) or with a truncated CP lacking eight amino acid residues from its N-terminal (AGII-HisΔ8; FIG. 1A; SEQ ID No.: 7). A cDNA containing AGII with a similar CP truncation but without an added peptide tag was constructed as a control for viral infectivity and systemic infection (AGIIΔ8, FIG. 1A; SEQ ID NO.: 1; Nucleotides: 25-837).

Both AGII-His and AGII-HisΔ8 (SEQ ID Nos.: 6 and 7) were 100% infectious on susceptible squash, melon and cucumber (Table 1). Symptoms appeared 7-8 dpi with similar characteristics to those of parental AGII virus. Likewise, all squash plants inoculated with AGIIΔ8 cDNA were systemically infectious. This result demonstrates that both an 8 AA deletion at the N-terminus of potyvirus CP and its substitution with a heterologous peptide are achievable.

Similar infectivity was obtained with histidine tagged cDNA constructs on cucumber and melon (Table 2). Both chimeric viruses were genetically stable in plants and kept the His-tag intact for at least 90 days and 3 subsequent passages in squash plants, as determined by RT-PCR of viral progeny and direct sequencing of the amplified product.

The accumulation of His-tagged CPs in systemically infected squash leaves was analyzed by immunoblot with an anti-His monoclonal antibody. A specific band was detected in AGII-His and AGII-HisΔ8 (SEQ ID Nos.: 6 and 7) extracts, but not in AGII or virus-free extract (FIG. 1B). A band with similar mobility was also detected by anti-CP polyclonal antibodies (FIG. 1B). Immunoblot with AB6, a monoclonal antibody that recognizes a specific heptapeptide in CP-NT (c.f. FIG. 1A; 7), showed that chimeric CPs accumulated to a similar level as wild type CP (FIG. 1B). In addition, the lower gel mobility of AGII-His CP is consistent with the predicted higher molecular weight resulting from the seven added amino acid residues.

Interestingly, DAS-ELISA of the above extracts with anti-CP antibodies failed to detect His-tagged virions (FIG. 1C). This is in agreement with the weak detection of His-tagged CPs by the same antibodies on western blot (FIG. 1B), suggesting that the protruding CP N-terminal domain containing most anti-CP epitopes (Desbiez, C., et al. (1997) J. Gen. Virol. 78:2073-2076) is masked by the exposed His-tag (FIG. 1C).

### Example 2

### Presentation of a heterologous peptide fused to the N-terminus of CP on the viral coat

In order to determine whether the fused His-tag (SEQ ID NOs.: 4 and 5) is exposed on the viral surface, virions were tested under native condition for their ability to bind to an Ni²⁺ affinity column. This column is known to bind exposed clusters of His residues (Schmitt, J., et al. (1993) Mol. Biol. Reports 18: 223-230). Soluble extracts from AGII-His (SEQ ID NO.: 6), AGII-HisΔ8 (SEQ ID NO.: 7) and AGII (SEQ ID NO.: 1) systemically infected squash leaves were subjected to Ni²⁺ affinity chromatography. Ni²⁺ binding virions were eluted with 300 mM imidazole. An equal volume from each fraction was analyzed by immunoblot. A protein of the same gel mobility as AGII CP was immuno-detected by anti-His antibody in the fractions eluted from AGII-His and AGII-HisΔ8 Ni²⁺ affinity columns (FIG. 2A, fractions E2-E5) but not in similar fractions from AGII Ni²⁺ affinity columns. Furthermore, no protein was detectable after excessive washes with 75 mM imidazole (FIG. 2A; lane wash). This demonstrates specific binding of His-tagged CPs to Ni²⁺.

In contrast, anti-CP antibodies detected non-tagged CP in similar amounts in the total (T) and flowthrough (Ft) fractions and not in the eluted fractions, indicating that native CP was present, although it does not bind Ni²⁺ (FIG. 2A; bottom panel). Electron microscopy analysis of AGII-His and AGII-HisΔ8 E4 fraction revealed intact virus particles structurally similar to AGII (FIG. 2B, lane E4). However, a higher proportion of broken particles was evident after purification treatments. In addition, these fractions were found to be infectious by mechanical inoculation experiments. In contrast, viral particles were not visible in AGII E4 fraction under the electron microscope, and its material was not infectious. Together these results prove that Ni²⁺ binding is done via the His-tag and demonstrate that the tag is exposed on AGII-His and AGII-HisΔ8 viral surfaces.

### Example 3

### Fusion of larger heterologous peptides to the N-terminus of CP

In order to determine whether a foreign peptide longer than seven amino acid residues would support virus assembly and systemic infection, a 48 nucleotide sequence encoding a sixteen amino acid peptide (SEQ ID NOs.: 8 and 9 respectively) from the human c-myc (Myc; 11) was cloned into the AGII genome to create a translational fusion with CP (AGII-Myc; FIG. 3A; SEQ ID NO.: 10). Recombinant AGII-Myc cDNA was able to infect cucurbits seedlings systemically as efficiently as AGII.

The AGII-Myc chimeric virus was genetically stable in plants and kept the Myc-tag intact for at least 60 days and 3 subsequent passages in squash plants, as determined by RT-PCR of viral progeny and direct sequencing of the amplified product. Accumulation of Myc-CP fusion protein in systemically infected squash leaves was analyzed by western blot analysis with anti-CP and anti-Myc antibodies. A band, with slightly slower gel mobility than AGII CP, was detected by anti-CP in AGII-Myc extract (FIG. 3B, top panel), as predicted from the fusion of Myc peptide to CP. This band was also specifically detected by Anti-Myc antibodies. A comparable level of CP was found in AGII-Myc and AGII by immunoblotting with AB6 monoclonal antibody (FIG. 3B). DAS-ELISA of the above extracts with anti-CP failed to detect chimeric virus, indicating steric hindrance similar to that described hereinabove for His-tagged viruses. These experiments show that the sixteen amino acid Myc tag can be successfully fused to the N-terminus of the potyvirus CP.

To further demonstrate the ability of an additional foreign sequence fused to the CP-NT to permit viral systemic infectivity, an eight AA sequence (residues 257-264 of ovalbumin; Porgador A, et al. (1997) J Immunol. 158, 834-41 [SEQ ID NOs.: 28 and 29]), was fused to the full-length CP-NT to create AGII-OVA ( SEQ ID NO.: 30; FIG. 7A).

Chimeric AGII-Ova cDNA (SEQ ID NO.: 30) were infectious on susceptible squash and symptoms appeared 7-8 days post inoculation. AGII-Ova viruses were genetically stable in plants and kept the fused sequence intact for at least 2 subsequent passages in squash plants, as determined by RT-PCR of viral progeny and direct sequencing of the amplified product. The accumulation of AGII-Ova in systemically infected leaves was verified by immunoblot with anti ZYMV-CP polyclonal antibodies. A specific band was detected in AGII-Ova with similar mobility to that of parental AGII suggesting that CP-Ova fusion protein accumulates in systemic leaves (FIG. 7B).

This data, together with data from example 2, establish that the exact size or sequence of the CP N-terminal domain is not essential for viral viability.

### Example 4

### A Heterologous Peptide Fused to an N-terminally Deleted CP Permits Viral Systemic Infection

In order to ascertain the necessity of the CP-NT domain for systemic infection of AGII, a systematic deletion analysis of CP-NT was conducted. Serial deletions were tested and the infectivity of mutant cDNAs was evaluated. Initially, AGII cDNAs containing a truncated CP-NT, lacking either 13 (AGIIΔ13, SEQ ID NO.: 27) or 33 (AGIIΔ33, SEQ ID NO.: 25) amino acid residues from its N-terminal, were constructed. Both cDNAs were found to be infectious, as was AGIIΔ8 (SEQ ID NO.: 26) cDNA (Table 2).

TABLE 2
Characteristics of AGII CP-NT mutants

| | SEQ | | Symptom | | |
|---|---|---|---|---|---|
| cDNA | ID | Infectivity | appearance^{b} | Virion | Systemic spread |
| clones | NO. | (%)^{a} | (dpi) | assembly^{c} | in cucurbits^{d} |
| AGII | 1 | 100 | 7 | + | S |
| AGIIΔ8 | 26 | 100 | 7 | + | S |
| AGIIΔ13 | 27 | 100 | 7 | + | S |
| AGIIΔ33 | 25 | 100 | 7 | + | S |
| AGII-His | 6 | 100 | 8 | + | S |
| AGII-HisΔ8 | 7 | 100 | 7 | + | S |
| AGII-Myc | 10 | 100 | 7 | + | S |
| AGII-MycΔ8 | 11 | 100 | 8 | + | S |
| AGII-MycΔ13 | 12 | 100 | 7 | + | S |
| AGII-MycΔ18 | 13 | 100 | 7 | + | S |
| AGII-MycΔ23 | 14 | 100 | 9 | + | S |
| AGII-MycΔ28 | 15 | 100 | 8 | + | S |
| AGII-MycΔ33 | 16 | 100 | 8 | + | S |
| AGII-MycΔ38 | 17 | 100 | 14 | + | S |
| AGII-MycΔ43 | 18 | 33 | 17 | + | S |
| AGII-MycΔ48 | 19 | 0 | − | NO | N |
| AGII-FMDV | 23 | 0 | − | NO | N |
| AGII-FMDVΔ13 | 22 | 0 | − | NO | N |
| AGII-Myc-FMDVΔ13 | 24 | 100 | 11 | + | S |
| ZYMV-MycΔ33 | 31 | 100 | 8 | + | S |
| AGII-Ova | 30 | 100 | 7-8 | + | S |

^{a}From a total of nine plants that were tested.
^{b}(−) no visible symptoms appeared for up to 30 dpi.
^{c}Virion particles were observed (+) or not (NO) under the electron microscope.
^{d}Systemic infection was observed (S) or not (N) in squash, melon and cucumber.

To further study whether the addition of a fused foreign peptide could maintain systemic infection of CP-NT-truncated AGII, serial deletions of CP-NT every five amino acids from position Ala₈ up to position Ala₄₈ (FIG. 4A) were prepared. The last deletion (AGIIΔ48, SEQ ID NO.: 1; Nucleotides 145-837) completely removed the 43-amino-acids-long CP-NT and part of the core. These deletions were then introduced into the AGII-Myc (SEQ ID NO.: 10) genome, to create a Myc translational fusion with truncated CPs (FIG. 4A; SEQ ID Nos.: 11-19). Mutated AGII cDNAs were tested for their ability to support systemic infection in planta. Squash seedlings were inoculated by particle bombardment with various constructs, and symptom appearance on non-inoculated leaves, indicative of systemic spread, was recorded.

As shown in Table 2, symptoms appeared 7-9 dpi as in the parental AGII, on plants inoculated with clones containing a deletion of up to 33 amino acid residues from the NT. Infectivity efficiency or symptom expressions were also unchanged. However, deletion of five more residues (AGII-MycΔ38; SEQ ID NO.: 17) delayed symptom appearance by 6 days, and deletion of an additional five (AGII-MycΔ43; Table 2) delayed it by nine days. Systemic infection of leaves with these two constructs impeded viral spread and caused milder symptoms than those infected by AGII and other mutant constructs, including His-tagged AGII.

In addition, AGII-MycΔ43 (SEQ ID NO.: 18) exhibited infectivity efficiency about three times lower than those of other infectious mutants (Table 2). It is noteworthy that deletion of up to 43 amino acids from CP-NT did not affect viral assembly, and the virus particles observed were indistinguishable from AGII particles under the electron microscope (FIG. 5; Data shown for AGII-MycΔ13, 23; SEQ ID Nos.: 12 and 14 respectively). No viral particles or symptoms were apparent in leaves after inoculation with AGII-MycΔ48 (SEQ ID NO.: 19). RT-PCR of viral progeny and direct sequencing of the amplified product confirmed the presence of the Myc sequence in the CP-NT of various deletion mutants. Immunoblot of total protein extracts from systemically infected squash leaves with anti-Myc monoclonal antibody detected a specific protein band in all Myc-tagged mutants and not in parental AGII (FIG. 4B). The increase in gel mobility of detected bands was consistent with the predicted reduced molecular weight of each deleted CP-NT. A similar level of Myc-CP fusion protein was detected in AGII-Myc, AGII-MycΔ8 and AGII-MycΔ13 (FIG. 4B; relative Myc-CP).

However, extended CP-NT truncations caused a stepwise decrease in the relative amount of Myc-CP fusion protein (FIG. 4B; relative Myc-CP). Protein bands with similar gel mobilities were also detected by anti-CP polyclonal antibody (FIG. 4B). In addition, immunoblot with AB6 monoclonal antibody, which recognizes CP-NT residues Gly₂₂ to Thr₂₈ (7; c.f. FIG. 1), detected chimeric CPs with truncations of up to 23 amino acid residues (FIG. 4B). No band was detected in CP-NT deletions greater than 23 amino acids, demonstrating that the expressed Myc-CP fusion protein did not contain the amino acid residues comprising the AB6-specific epitope (FIG. 4B). Furthermore, weak detection of AGII-MycΔ23 (SEQ ID NO.: 14) CP by AB6 antibody is consistent with the loss of two amino acid residues from the AB6 epitope and does not reflect AGII-MycΔ23 CP accumulation level. To verify that these results were not unique to the AGII, an attenuated ZYMV (Gal-On (2000) Phytopathology 90:467-473), a ZYMV-MycΔ33 (SEQ ID NO.: 31) clone was constructed as was described for AGII-MycΔ33 (SEQ ID NO.: 16). Infection of squash seedlings with ZYMV-MycΔ33 resulted in systemic infection with similar characteristics to those of AGII-MycΔ33 (Table 1). As expected, use of this construct produced more severe disease symptoms. Immunoblot analysis of leaves verified the presence of chimeric CP with similar gel mobility to that of AGII-MYCΔ33? (FIG. 4C).

These results demonstrate for the first time that the entire CP-NT of ZYMV-AGII sequence can be deleted or replaced by a non-viral sequence while maintaining viral systemic infection.

### Example 5

### Presentation of Myc Peptide Fused to the CP N-terminus on the Viral Surface.

In order to establish that the CP-Myc fusion leads to presentation of the Myc tag on the viral surface, quantitative DAS-ELISA of samples with anti-Myc antibody was performed using samples taken from the same leaves used for the immunoblot analysis of example 4. An ELISA signal was detected with all mutant virus samples, but not with the AGII sample.

This demonstrates that the Myc epitope is exposed on the chimeric viral surface (FIG. 5A). Interestingly, stronger signals were apparent with AGII-MycΔ38 and Δ43 (SEQ ID NOs.: 17 and 18 respectively) despite the fact that CP accumulation does not seem to be higher using these strains according to immunoblot results (FIG. 4B, relative Myc-CP). This indicates that the Myc peptide displayed on a CP with a deleted N-terminal domain is more accessible to anti-Myc antibody than in other mutants containing longer N terminal domains. (FIG. 5A).

Immunogold labeling experiments were performed comparing representative chimeric viruses and AGII. The morphology of all virus particles was similar (FIG. 6). AGII-Myc, AGII-MycΔ13 and AGII-MycΔ23 (SEQ ID NOs.: 10, 12 and 14) were successfully gold-labeled when incubated with a monoclonal antibody against Myc. In contrast, no labeling was apparent with AGII.

Together, these data provide conclusive evidence that the Myc peptide fused to intact or truncated CP-NT is presented on the viral surface so that its antigenic determinants are exposed.

### Example 6

### Restoration of Systemic Infectivity of AGII with FMDV Peptide Fused to its CP-NT by Additional Fusion of a Myc Tag.

In order to assess the ability of other foreign sequences to permit systemic infectivity of AGII mutants with deletions in the N-terminal domain of CP, the 16 amino acid long foot and mouth disease virus coat protein immunogenic epitope (FMDV (SEQ ID NOs.: 20 and 21) was fused to AGIIΔ13 (AGII-FMDVΔ13; SEQ ID NO.: 24; FIG. 6A). As a positive control, the FMDV was fused to AGII CP to create AGII-FMDV (SEQ ID NO.: 23; FIG. 6A).

Both cDNA clones were non-infectious. This indicated that FMDV disrupts viral infectivity. The possibility of restoring viral infectivity by additional fusion of Myc upstream of the FMDV was tested. The sequence encoding the FMDV peptide was inserted into the AGII-MycΔ13 (SEQ ID NO.: 12) to create a translational fusion with Myc at the N-terminus. This created a 31 amino acid heterologous peptide fused to CPΔ13 NT designated AGII-Myc-FMDVΔ13 (FIG. 6A; SEQ ID NO.: 24).

This new clone was infectious on various cucurbits and typical symptoms appeared with a delay of 2-3 days compared to AGII (Table 2). The AGII-Myc-FMDVΔ13 virus was genetically stable in plants and kept Myc and FMDV sequences intact for at least 30 days or three subsequent passages in squash plants, as determined by RT-PCR of viral progeny and direct sequencing of the amplified product. Immunoblot analysis of squash leaf extracts with either anti-Myc monoclonal antibody or anti-FMDV and anti-CP polyclonal antibodies detected a protein band with a similar mobility, verifying that both tags are fused to the same coat protein (FIG. 6B; lane AGII-Myc-FMDVΔ13). In contrast, anti-FMDV antibody did not detect any band in extracts from AGII or AGII-MycΔ13 (FIG. 6B). DAS-ELISA assay of the above extracts using anti-Myc or anti-FMDV antibodies detected both tags (FIG. 6C). Only background signal was detected when AGII-MycΔ13 was probed with anti-FMDV antibodies.

These results demonstrate that both Myc and FMDV epitopes are exposed on the surface of AGII-Myc-FMDVΔ13 virions. Comparison of predicted isoelectric point (pI) between wild type CP and various chimeric CPs (Table 3), shows that viral systemic infectivity was compromised only in cases where a relatively strong basic change in chimeric CP pI was caused by fusion of a heterologous peptide (AGII-FMDV & AGII-FMDVΔ13; Table 3). Fusion of an acidic Myc peptide (AGII-Myc-FMDVΔ13) that neutralizes the basic isoelectric point of non-infectious AGII-FMDVΔ13 CP restored viral systemic infectivity (Table 3). These results suggest that maintaining a near neutral CP isoelectric point is important for viral systemic infectivity.

TABLE 3
Charge value of chimeric CP for successful epitope display

| | | | Predicted pI | |
|---|---|---|---|---|
| | Systemic | Size of fused | of chimeric | Δ charge from |
| Viral construct | infectivity | peptide (aa) | CP | w.t. CP |
| AGII | + | 0 | 6.66 | − |
| AGII-His | + | 7 | 6.89 | +0.23 |
| AGII-Myc | + | 16 | 6.02 | −0.64 |
| AGII-Ova | + | 8 | 6.71 | +0.05 |
| AGII-Oral | + | 20 | 6.45 | −0.21 |
| AGII-FMDV | − | 17 | 8.76 | +2.10 |
| AGII-FMDVΔ13 | − | 17 | 8.45 | +1.79 |
| AGII-Myc-FMDVΔ13 | + | 31 | 7.05 | +0.39 |

### Example 7

### Oral and Intranasal Application of Partially Purified AGII-Ova Virions to Mice Produces an Immune Response against ZYMV CP

The AGII-Ova (SEQ ID NO.: 30) chimeric virion was employed to test the immunogenicity of ZYMV CP by combined oral and nasal administration of virions to mice. Partially purified virions in borate buffer (5.6 μg/μl) were orally (20 μl) and intra-nasally (20 μl ) administrated to a group of three mice. As a control the same volume of borate buffer was administrated to a control mouse. A second administration intra-nasally of 20 μl of virions or buffer was preformed after 16 days. Serum was collected 35 days later from control and challenged mice.

Each serum sample was then tested for the presence of anti ZYMV CP antibodies by immunoblot analysis against AGII coat protein. As shown in FIG. 7C, a specific signal was observed in all challenged mice but not in the control mouse verifying the presence of anti-CP antibodies in the mice serum. These results establish that nasal or oral application of CP immunogenic epitopes assembled as a virion cause an immune response in vivo.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A recombinant vector for expressing a heterologous peptide at the amino-terminus of a potyvirus coat protein, the vector comprising;
(a) sufficient potyvirus nucleic acid sequence to permit viral replication and spread within a plant infected by the vector; and
(b) a heterologous nucleic acid sequence inserted at the amino-terminus of the potyvirus coat protein.

2. The recombinant vector of claim 1, wherein the amino-terminus is selected from the group consisting of:
(i) an established amino-terminus of a wild type potyvirus coat protein; and
(ii) an alternate amino-terminus of a potyvirus coat protein, said alternate amino-terminus arising from an action selected from the group consisting of an insertion, a replacement and a deletion of at least one amino acid residue from said known amino-terminus.

3. The recombinant vector of claim 1, wherein said heterologous nucleic acid sequence encodes at least a portion of the heterologous peptide.

4. The recombinant vector of claim 1, wherein the potyvirus is zucchini yellow mosaic virus (ZYMV).

5. The recombinant vector of claim 1, wherein the potyvirus is selected from the group consisting of ALMV, AmLMV, ArjMV, ArLV, AV-1, BCMV, BCNMV, BYMV, BtMV, BiMoV, CdMV, CVMV, CTLV, CeMV, ChiVMV, CIYVV, CSV, CDV, ComMV, CABMV, CGVBV, DsMV, DSTV, DeMV, GSMV, GEV, GGMV, HVY, HMV, HiMV, IFMV, IMMV, ISMV, JGMV, KMV, LYSV, LMV, MDMV, NDV, NYSV, NoMV, OYDV, ORMV, BRSV, PARMV, PWV, PSBMV, PEMOV, PEPMOV, PESMV, PVMV, PTV, PPV, PKMV, PVA, PVV, PVY, RETBV, SRMV, SMV, SCMV, SPFMV, TAMMV, TEMV, TEV, TVMV, TBBV, TBV, TSBV, TUMV, WMV-2, WVMV, YMV and ZYFV.

6. The recombinant vector of claim 1, wherein said potyvirus nucleic acid sequence and said heterologous nucleic acid sequence are each selected from the group consisting of a DNA sequence, an RNA sequence a cDNA sequence and combinations thereof.

7. The recombinant vector of claim 1, wherein the coat protein of the potyvirus includes an amino-terminal domain.

8. The recombinant vector of claim 7, wherein said amino-terminal domain is modified by deletion of at least 1 amino acid residue.

9. The recombinant vector of claim 3, wherein fusion to the potyvirus coat protein influences a biological activity of said at least a portion of the heterologous peptide.

10. The recombinant vector of claim 3, wherein said at least a portion of a heterologous peptide is selected from the group consisting of an antigen, a receptor, a ligand, an enzyme, a surfactant, a pore forming molecule, a chaperone, a nucleic acid binding molecule, a transcription factor, an inhibitor, an ion binding molecule, a carbohydrate binding molecule and a signal transducer.

11. The recombinant vector of claim 3, wherein said at least a portion of the heterologous peptide is capable of eliciting an immune response when appropriately administered to an animal.

12. The recombinant vector of claim 11, wherein said animal is selected from the group consisting of an insect, a fish, a bird, a reptile and a mammal.

13. The recombinant vector of claim 12, wherein said mammal is selected from the group consisting of a house pet, a laboratory animal, a sheep, a goat, a cow, a pig, a monkey, an ape and a human being.

14. The recombinant vector of claim 1, further comprising an amino acid substitution in the HC-Pro gene of the conserved FRNK box of said potyvirus nucleic acid sequence, said substitution causing attenuation of the potyvirus.

15. The recombinant vector of claim 1, further comprising an amino acid substitution in said potyvirus nucleic acid sequence, said substitution effectively abolishing aphid transmissibility of the potyvirus.

16. The recombinant vector of claim 1, wherein said heterologous nucleic acid sequence encodes at least a portion of a peptide selected from the group consisting of cMYC, FMDV, His tag and Ovalbumin.

17. The recombinant vector of claim 16, wherein said heterologous nucleic acid sequence comprises at least a portion of at least one member selected from the group consisting of SEQ ID NOs.: 4, 8, 20 and 28.

18. The recombinant vector of claim 1, wherein said heterologous nucleic acid sequence encodes at least a portion of at least one peptide selected from the group consisting of SEQ ID NOs.: 5, 9, 21 and 29.

19. The recombinant vector of claim 1, wherein the vector comprises at least a portion of at least one item selected from the group consisting of SEQ ID Nos.: 6, 7, 10-19, 22-27, 30 and 31.

20. The recombinant vector of claim 1, further comprising one additional amino acid residue, said additional amino acid residue facilitating proteolytic excision of the coat protein from within a potyvirus polyprotein.

21. The recombinant vector of claim 20, wherein said additional residue is selected from the group consisting of serine, methionine, glycine, alanine and phenylalanine.

22. The recombinant vector of claim 1, wherein translation of said potyvirus nucleic acid sequence encoding a portion of an amino-terminal domain of a potyvirus coat protein and said heterologous nucleic acid sequence fused thereto produces a fusion protein with an isoelectric point similar to an isoelectric point of a native potyvirus coat protein.

23. A method of transiently expressing at least a portion of a heterologous peptide in at least a portion of a plant, the method comprising the steps of:
(a) providing a recombinant vector comprising;
(i) sufficient potyvirus nucleic acid sequence to permit viral replication and spread within a plant infected by the vector;
(ii) a heterologous nucleic acid sequence inserted at the amino-terminus of the potyvirus coat protein;
(b) introducing at least one copy of said vector into at least one cell of the plant; and
(c) cultivating the plant so that said vector is transcribed, the resulting nucleic acid replicating therein by forming viable potyvirus virions which spread from cell to cell within the plant;
wherein each of said viable potyvirus virions displays on an external surface thereof a plurality of copies of the at least a portion of the heterologous peptide.

24. The method of claim 23, wherein the vector is transcribed extrachromosomally.

25. The method of claim 23, wherein the plant is a cucurbit plant.

26. A plant transiently expressing at least a portion of a heterologous peptide in at least a portion thereof, the plant comprising:
(a) at least one cell infected with a recombinant vector for expressing the at least a portion of the heterologous peptide, the vector comprising;
(i) sufficient potyvirus nucleic acid sequence to permit viral replication and spread within the plant infected by the vector;
(ii) a heterologous nucleic acid sequence inserted at the amino-terminus of the potyvirus coat protein
wherein said vector is transcribed extrachromosomally, thereby forming viable potyvirus virions capable of replicating and spreading from cell to cell within the plant; and
wherein each of said viable potyvirus virions displays on an external surface thereof a plurality of copies of said at least a portion of a heterologous peptide.

27. The plant of claim 26, wherein the plant is a cucurbit plant.

28. A method of vaccination, the method comprising the steps of:
(a) providing a recombinant vector comprising;
(i) sufficient potyvirus nucleic acid sequence to permit viral replication and spread within a plant infected by the vector;
(ii) a heterologous nucleic acid sequence inserted at the amino-terminus of the potyvirus coat protein, wherein said heterologous nucleic acid sequence encodes at least one antigenic determinant;
(b) introducing at least one copy of said vector into at least one cell of a plant;
(c) cultivating said plant so that said vector is transcribed therein resulting in the formation of viable potyvirus virions which replicate and spread from cell to cell within said plant;
wherein each of said potyvirus virions displays on an external surface thereof a plurality of copies of said at least one antigenic determinant;
(d) harvesting at least a portion of said plant; and
(e) delivering said potyvirus virions to a subject, such that said plurality of copies of said at least one antigenic determinant contained therein elicit an immune response from said subject.

29. The method of claim 28, comprising the additional step of isolating said potyvirus virions from said at least a portion of said plant.

30. The method of claim 28, wherein said step of delivering includes oral administration to said subject.

31. The method of claim 28, wherein said step of delivering and said step of harvesting are carried out concurrently.

32. The method of claim 28, wherein said step of delivering is accomplished by a means selected from the group consisting of injection, oral administration, intraocular administration, intranasal administration, transdermal delivery, aerosol delivery, intravaginal administration and rectal administration.

33. The method of claim 28, wherein said plant is a cucurbit plant.

34. The method of claim 28, wherein said vector replicates extrachromosomally.

35. The method of claim 28, wherein said vector replicates outside a nucleus of said at least one cell of said plant.


SEQUENCE LISTING (33 sequences)

SEQ ID NO: 1 - DNA, 837 bp, Zucchini yellow mosaic virus
tcaggcactcagccaactgtggcagatgctggagctacaaagaaagataaagaagatgacaaagggaaaaacaaggacgttacaggctccggctcaggtgagaaaacagtagcagctgtcacgaaggacaaggatgtgaatgctggttctcatgggaaaattgtgccgcgtctttcgaagatcacaaagaaaatgtcattgccacgcgtgaaaggaaatgtgatactcgatattgatcatttgctggaatataaaccggatcaaattgagttatataacacacgagcgtctcatcagcagttcgcctcttggttcaaccaggttaagacggaatatgatttgaacgagcaacagatgggagttgtaatgaatggtttcatggtttggtgcattgagaatggcacttcacccgacattaatggagtgtgggttatgatggacggaaatgagcaagttgagtatcccttgaaaccaatagttgaaaatgcaaagccaacgctgcggcaaataatgcatcatttttcagatgcagcggaggcatatatagagatgagaaatgcagaggcaccatacatgccgaggtatggtttgcttcgaaacctacgggataggagtttagcacgatatgcttttgatttctatgaagtcaattctaaaactcctgaaagagcccgcgaagctgttgcgcagatgaaagcagcagctcttagcaatgtttcttcaaggttgtttggccttgatggaaatgttgccaccactagcgaagacactgaacggcacactgcacgtgatgttaatagaaacatgcacaccttactaggtgtgaatacaatgcag

SEQ ID NO: 2 - protein, 279 aa, Zucchini yellow mosaic virus
SGTQPTVADAGATKKDKEDDKGKNKDVTGSGSGEKTVAAVTKDKDVNAGSHGKIVPRLSKITKKMSLPRVKGNVILDIDHLLEYKPDQIELYNTRASHQQFASWFNQVKTEYDLNEQQMGVVMNGFMVWCIENGTSPDINGVWVMMDGNEQVEYPLKPIVENAKPTLRQIMHHFSDAAEAYIEMRNAEAPYMPRYGLLRNLRDRSLARYAFDFYEVNSKTPERAREAVAQMKAAALSNVSSRLFGLDGNVATTSEDTERHTARDVNRNMHTLLGVNTMQ

SEQ ID NO: 3 - DNA, 20 bp, Zucchini yellow mosaic virus
catttcctttcacgcgtggc

SEQ ID NO: 4 - DNA, 21 bp, Artificial Sequence
  Nucleotide sequence encoding Hexa-Histidine with a Serine at its N′
tcacaccatcaccatcaccat

SEQ ID NO: 5 - protein, 7 aa, Artificial Sequence
  Hexa-Histidine (His) peptide fuse to serine (Ser) at its N′
SHHHHHH

SEQ ID NO: 6 - DNA, 53 bp, Artificial Sequence
  Nucleotide sequence encoding Hexa-histidine fused to the N-termin us of Zucchini Yellow Mosaic Virus coat protein gene
cagctgcagtcacaccatcaccatcaccattcaggcactcagccaactgtggc

SEQ ID NO: 7 - DNA, 55 bp, Artificial Sequence
  Nucleotide sequence encoding Hexa-histidine fused to the N-termin us of Zucchini Yellow Mosaic Virus coat protein gene of which the N′ terminal 8 amino acids were deleted
cagctgcagtcacaccatcaccatcaccatgatactggagctacaaagaaagaag

SEQ ID NO: 8 - DNA, 45 bp, Homo sapiens
tcagcatcagagcagaagctcatttcagaggaggatctcggatcc

SEQ ID NO: 9 - protein, 15 aa, Homo sapiens
SASEQKLISEEDLGS

SEQ ID NO: 10 - DNA, 77 bp, Artificial Sequence
  Nucleotide sequence encoding the peptide derived from Human c-Myc-tag fused to the N-terminus of Zucchini Yellow Mosaic Virus (AGII) coat protein gene
cagctgcagtcagcatcagagcagaagctcatttcagaggaggatctcggatcctcaggcactcagccaactgtggc

SEQ ID NO: 11 - DNA, 82 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 8 amino acids were deleted
cagctgcagtcagcatcagagcagaagctcatttcagaggaggatctcggatccgatactggagctacaaagaaagataaag

SEQ ID NO: 12 - DNA, 81 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 13 amino acids were deleted
cagctgcagtcagcatcagagcagaagctcatttcagaggaggatctcggatccaagaaagataaagaagatgacaaaggg

SEQ ID NO: 13 - DNA, 31 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 18 amino acids were deleted
cgcggatccgatgacaaagggaaaaacaagg

SEQ ID NO: 14 - DNA, 30 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 23 amino acids were deleted
ctcggatccaacaaggatgttacaggctcc

SEQ ID NO: 15 - DNA, 27 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 28 amino acids were deleted
cgcggatccggctccggctcaagtgag

SEQ ID NO: 16 - DNA, 30 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 33 amino acids were deleted
cgcggatccgagaaaacagtggcagctgtc

SEQ ID NO: 17 - DNA, 28 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 38 amino acids were deleted
cgcggatccgctgtcacgaaggacaagg

SEQ ID NO: 18 - DNA, 33 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 43 amino acids were deleted
cgcggatccaaggatgtaaatgctggttctcat

SEQ ID NO: 19 - DNA, 30 bp, Artificial Sequence
  Nucleotide sequence encoding the peptide derived from a partial Human c-Myc-tag fused to the N-terminus of Zucchini Yellow Mosaic Virus (AGII) coat protein gene of which the N′ terminal 48 amino acids were deleted
ctcggatccggttctcatgggaaaattgtg

SEQ ID NO: 20 - DNA, 51 bp, Foot-and-mouth disease virus
agtgtgagaggagatcttcaagtgcttgcacgaaaagcagcaagaccactt

SEQ ID NO: 21 - protein, 17 aa, Foot-and-mouth disease virus
SVRGDLQVLARKAARPL

SEQ ID NO: 22 - DNA, 87 bp, Artificial Sequence
  Nucleotide sequence encoding the peptide derived from the Foot and Mouse disease Virus (FMDV) coat protein epitope fused to the N-terminus of Zucchini Yellow Mosaic Virus (AGII) coat protein gene of which the N′ terminal 13 amino acids were deleted
cagctgcagtccgtgagaggagatcttcaagtgcttgcacgaaaagcagcaagaccacttaagaaagataaagaagatgacaaaggg

SEQ ID NO: 23 - DNA, 83 bp, Artificial Sequence
  Nucleotide sequence encoding the peptide derived from the Foot and Mouse disease Virus (FMDV) coat protein epitope fused to the N-terminus of Zucchini Yellow Mosaic Virus (AGII) coat protein gene
cagctgcagagtgtgagaggagatcttcaagtgcttgcacgaaaagcagcaagaccactttcaggcactcagccaactgtggc

SEQ ID NO: 24 - DNA, 84 bp, Artificial Sequence
  Nucleotide sequence encoding the peptide derived from a partial Human c-Myc-tag fused to the N-terminus of FMDV coat protein epitope fused to N-terminus of Zucchini Yellow Mosaic Virus (AGII) coat protein gene of which N′ terminal 13 aa′s were deleted
cgcggatccgtgagaggagatcttcaagtgcttgcacgaaaagcagcaagaccacttaagaaagataaagaagatgacaaaggg

SEQ ID NO: 25 - DNA, 33 bp, Artificial Sequence
  Nucleotide sequence encoding the Zucchini Yellow Mosaic Virus (ZYMV-AGII) NIa protease motif fused to the N- terminus  of ZYMV (AGII) coat protein gene of which the N′ terminal 33 amino acids were deleted
cagctgcagtccgagaaaacagtggcagctgtc

SEQ ID NO: 26 - DNA, 34 bp, Artificial Sequence
  Nucleotide sequence encoding the Zucchini Yellow Mosaic Virus (ZYMV-AGII) NIa protease motif fused to the N- terminus of ZYMV (AGII) coat protein gene of which the N′ terminal 8 amino acids were deleted
cagctgcagtccgacactggagccacaaagaaag

SEQ ID NO: 27 - DNA, 37 bp, Artificial Sequence
  Nucleotide sequence encoding the Zucchini Yellow Mosaic Virus (ZYMV-AGII) NIa protease motif fused to the N- terminus of ZYMV (AGII) coat protein gene of which the N′ terminal 13 amino acids were deleted
cagctgcagtccaagaaagacaaagaagatgacaaag

SEQ ID NO: 28 - DNA, 24 bp, Gallus gallus
tccattattaatttcgaaaagttg

SEQ ID NO: 29 - protein, 8 aa, Gallus gallus
SIINFEKL

SEQ ID NO: 30 - DNA, 56 bp, Artificial Sequence
  Nucleotide sequence encoding the peptide derived from the Ovalbum in residues 257-264 fused to the N- terminus of Zucchini Yellow Mosaic Virus (AGII) coat protein gene
cagctgcagtccattattaatttcgaaaagttgtcaggcactcagccaactgtggc

SEQ ID NO: 31 - DNA, 33 bp, Artificial Sequence; identical to SEQ ID NO: 25
  Nucleotide sequence encoding the peptide derived from a partial Human c-Myc-tag fused to the N-terminus of Zucchini Yellow Mosaic Virus coat protein gene of which the N′ terminal 33 amino acids were deleted

SEQ ID NO: 32 - DNA, 22 bp, Zucchini yellow mosaic virus
agctccatacatagctgagaca

SEQ ID NO: 33 - DNA, 20 bp, Zucchini yellow mosaic virus
tggttgaaccaagaggcgaa